(19) United States
(12) Patent Application Publication
(10) Pub. No.: US 2015/0038691 A1
(43) Pub. Date: Feb. 5, 2015

(54) POLYNUCLEOTIDE CONFIGURATION FOR RELIABLE ELECTRICAL AND OPTICAL SENSING

(71) Applicant: INTERNATIONAL BUSINESS MACHINES CORPORATION, Armonk, NY (US)

(72) Inventors: Binquan Luan, Chappaqua, NY (US); Ajay K. Royyuru, Congers, NY (US); Gustavo A. Stolovitzky, Riverdale, NY (US); Chao Wang, Ossining, NY (US); Deqiang Wang, Ossining, NY (US)

(73) Assignee: INTERNATIONAL BUSINESS MACHINES CORPORATION, Armonk, NY (US)

(21) Appl. No.: 14/012,212

(22) Filed: Aug. 28, 2013

Related U.S. Application Data
(63) Continuation of application No. 13/956,033, filed on Jul. 31, 2013.

(51) Int. Cl. C12N 15/11

(52) U.S. Cl. CPC C12N 15/11
USPC 536/25.32; 536/25.3

(57) ABSTRACT

A mixed polynucleotide includes a first double stranded (ds) portion, a second portion including at least one single stranded (ss) portion, and a third ds portion. The second portion connects the first ds portion and the third ds portion to provide a modified polynucleotide.

### RELATED APPLICATION DATA

This application is a Continuation application of co-pending U.S. patent application Ser. No. 13/956,033 filed on Jul. 31, 2013, incorporated herein by reference in its entirety.

### BACKGROUND

1. Technical Field

The present invention relates to polynucleotide configurations, and more particularly to polynucleotide configurations with mixed single-stranded and double-stranded segments for reliable electrical and optical sensing.

2. Description of the Related Art

Accurate and inexpensive sensing of nucleic acids (e.g., DNA and RNA) is important to understanding many scientific and biomedical applications. Solid-state bio-sensing techniques, such as artificial nanopores and channels, have been integrated into fluidics for sensing of polynucleotide molecules. Recently, tunneling recognition in a nanofluidic device has been shown to be a promising technology for next-generation low-cost and ultrafast DNA sequencing.

While using different device configurations, these solid-state sensing technologies rely on an ultra-confined nanofluidic chamber (a channel or a pore) with critical dimensions down to sub-10 nm to achieve the necessary linearization and hence sequential base reading of a single stranded polynucleotide. Because of its small persistence length (3-5 nm), single stranded polynucleotide molecules are very flexible to coil into various conformations outside the nanochannel or nanopore, and they experience a very high entropic energy barrier (the energy needed to decoil) to translocate. This usually results in a decreased translocation rate, undesirable polynucleotide bouncing-and-retreating rather than translocation, translocation with folded configuration, and long-time clogging of a nanochannel or a nanopore.

### SUMMARY

A mixed polynucleotide includes a first double stranded (ds) portion, a second portion including at least one single stranded (ss) portion, and a third ds portion. The second portion connects the first ds portion and the third ds portion to provide a modified polynucleotide.

A method for forming a polynucleotide including forming sticky ends on at least one end of a plurality of double stranded (ds) polynucleotide segments and one or more single stranded (ss) polynucleotide segments. The sticky ends of the one or more ss polynucleotide segments are joined between the plurality of ds polynucleotide segments to provide a mixed ds-ss polynucleotide

A method for forming a polynucleotide includes determining a plurality of recognition sites on a double stranded (ds) polynucleotide segment. The ds polynucleotide segment is cut at the plurality of recognition sites to form at least one single stranded (ss) polynucleotide segment between ds polynucleotide segments to provide a mixed ds-ss polynucleotide.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1A shows a stretched single stranded polynucleotide, in accordance with one illustrative embodiment;

FIG. 1B shows a coiled single stranded polynucleotide, in accordance with one illustrative embodiment;

FIG. 1C shows double stranded polynucleotide segments, in accordance with one illustrative embodiment;

FIG. 2A shows a mixed double stranded and single stranded polynucleotide, in accordance with one illustrative embodiment;

FIG. 2B shows a mixed polynucleotide having double stranded ends and a plurality of double stranded and single stranded polynucleotide segments therebetween, in accordance with one illustrative embodiment;

FIG. 3A shows a formation of a mixed polynucleotide by ligation, in accordance with one illustrative embodiment;

FIG. 3B shows a formation of a mixed polynucleotide by cutting, in accordance with one illustrative embodiment;

FIG. 3C shows a formation of a mixed polynucleotide by random sequencing, in accordance with one illustrative embodiment;

FIG. 4A shows a single stranded polynucleotide in a volume with a fixed diameter D in the range of 5-50 nm, in accordance with one illustrative embodiment;

FIG. 4B shows a mixed polynucleotide in a volume with a fixed diameter D in the range of 5-50 nm, in accordance with one illustrative embodiment;

FIG. 5A shows a single stranded polynucleotide in a volume having varying regions of confinement (50 nm>D1>D2>5 nm), in accordance with one illustrative embodiment;

FIG. 5B shows a graph of electrostatic energy and Gibbs free energy of the single stranded polynucleotide of FIG. 5A, in accordance with one illustrative embodiment;

FIG. 5C shows a double stranded polynucleotide in a volume having varying regions of confinement (50 nm>D1>D2>5 nm), in accordance with one illustrative embodiment;

FIG. 5D shows a graph of electrostatic energy and Gibbs free energy of the mixed polynucleotide of FIG. 5C, in accordance with one illustrative embodiment;

FIG. 6 shows electrical detection of a mixed nucleotide, in accordance with one illustrative embodiment;

FIG. 7A shows a single stranded polynucleotide chain in a volume having trapping electrodes, in accordance with one illustrative embodiment;

FIG. 7B shows a mixed polynucleotide chain in a volume being pushed by electrostatic force, in accordance with one illustrative embodiment;

FIG. 8A shows a mixed polynucleotide labeled with a fluorescent dye, in accordance with one illustrative embodiment;

FIG. 8B shows a mixed polynucleotide labeled with two different fluorescent dyes that emit different colors by binding to single stranded and double stranded segments, in accordance with one illustrative embodiment; and

FIG. 9 shows a block/flow diagram showing a system/method for forming a mixed single stranded and double stranded polynucleotide, in accordance with one illustrative embodiment.

### DETAILED DESCRIPTION OF PREFERRED EMBODIMENTS

In accordance with the present principles, polynucleotide configurations and methods of formation are provided. A polynucleotide chain is provided having mixed single stranded and double stranded segments for multi-functional sensing. The polynucleotide chain has single stranded segments for single-nucleotide electronic reading and double stranded segments for improved motion control, enhanced electronic reading rate, and boosted fluorescence imaging quality. Electrical and optical measurement methods using nanofluidic devices for detection of such polynucleotide molecules are also provided.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It will also be understood that when an element such as a layer, region or substrate is referred to as being “on” or “over” another element, it can be directly on the other element or intervening elements may also be present. In contrast, when an element is referred to as being “directly on” or “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

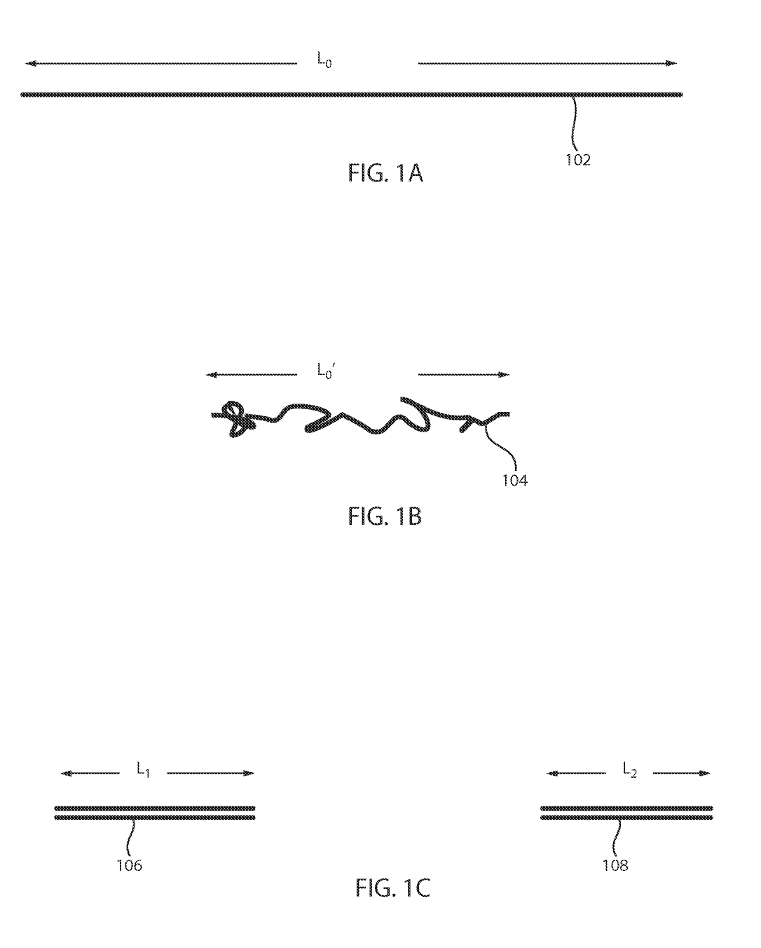

Referring now to the drawings in which like numerals represent the same or similar elements and initially to FIGS. 1A and 1B, single stranded (ss) polynucleotide chains are illustratively depicted in accordance with one illustrative embodiment. The ss polynucleotide chain may include, e.g., a DNA chain or an RNA chain. The ss polynucleotide chain 102 is in a linear stretched state with a contour length of L₀ and N₀ base pairs. In general, ss polynucleotide chains do not assume a linear state, but rather a relaxed state in a coiled configuration, such as ss polynucleotide chain 104. This is because an ss polynucleotide has a persistence length of about, e.g., 2-5 nm, and it can be stretched in the ultra-narrow nanochannel or nanopore for a few nanometers, which are extremely hard to fabricate. The ss polynucleotide chain 104 has a length L₀′, which is dependent on geometrical confinement.

A nanochannel and a nanopore refer to a one-dimensional volume with its depth and width well within the nanoscale (e.g., from a few nanometers to 100 nm), while its length may be much larger (e.g., tens of nanometers to micrometers). Specifically, a nanochannel refers to a nano-confined volume on a planar surface with the top-sealed, while a nanopore refers to a nano-sized hole vertically drilled through a membrane.

Referring now to FIG. 1C, a double stranded (ds) polynucleotide chain is illustratively depicted in accordance with one embodiment. A ds polynucleotide chain may include, e.g., a DNA chain or a DNA-RNA complementary chain. The ds polynucleotide chain may include segments, such as segments 106 and 108 having contour lengths L₁ and L₂, respectively. In contrast to ss polynucleotide chains, it is much less stringent to stretch ds polynucleotide chains. This is because its persistence length is much larger, around, e.g., 50 nm or 150 base pair (bp). This means much less stringent fabrication techniques may be used to create a channel or a nanopore to fully stretch the ds polynucleotide chain, as long as its dimensions are smaller than about, e.g., 50 nm.

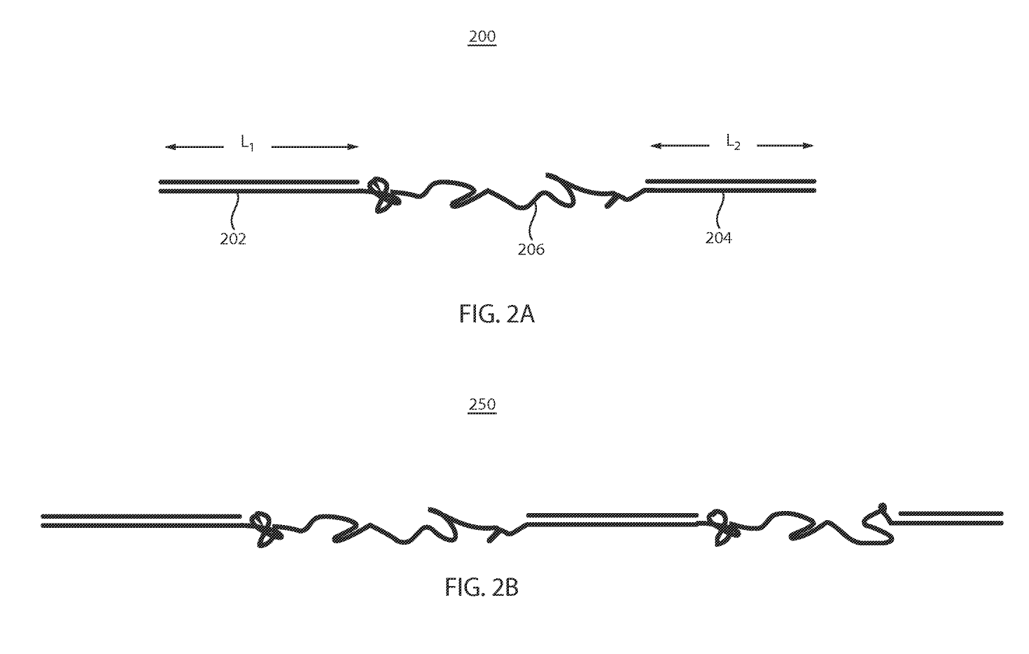

Referring now to FIG. 2A, a hybrid polynucleotide chain 200 is illustratively depicted in accordance with one embodiment. The hybrid polynucleotide chain 200 includes mixed ds and ss segments. In one embodiment, the ds-ss hybrid polynucleotide chain 200 includes ds segments 202, 204 at the ends of the chain, and an ss segment 206 in the middle. The ss segment 206 may include the bases to be detected and the ds segments 202, 204 provide the necessary rigidity for better control of the polynucleotide motion.

To minimize the flexibility of the ds segments 202, 204, the lengths of the segments L₁ and L₂, respectively, can be controlled to be smaller than, close to, or slightly larger than the Kuhn length (twice the persistence length), or about, e.g., 100 nm. The corresponding base pair numbers N₁ and N₂ for ds segments 202, 204, respectively, are accordingly on the order of 300. Within such a length scale, the ds segments 202, 204 behave like reasonably rigid rods and do not coil. Preferably, L₁ and L₂ are smaller than the Kuhn length such that the ds segments 202, 204 are always linear and have a very small energy barrier for translocation.

Referring now to FIG. 2B, a hybrid polynucleotide chain 250 is shown in accordance with one illustrative embodiment. The hybrid polynucleotide chain 250 may include a plurality of ss segments and/or a plurality of ds segments in the middle of the chain 250. This allows for continuous reading of longer polynucleotide chains.

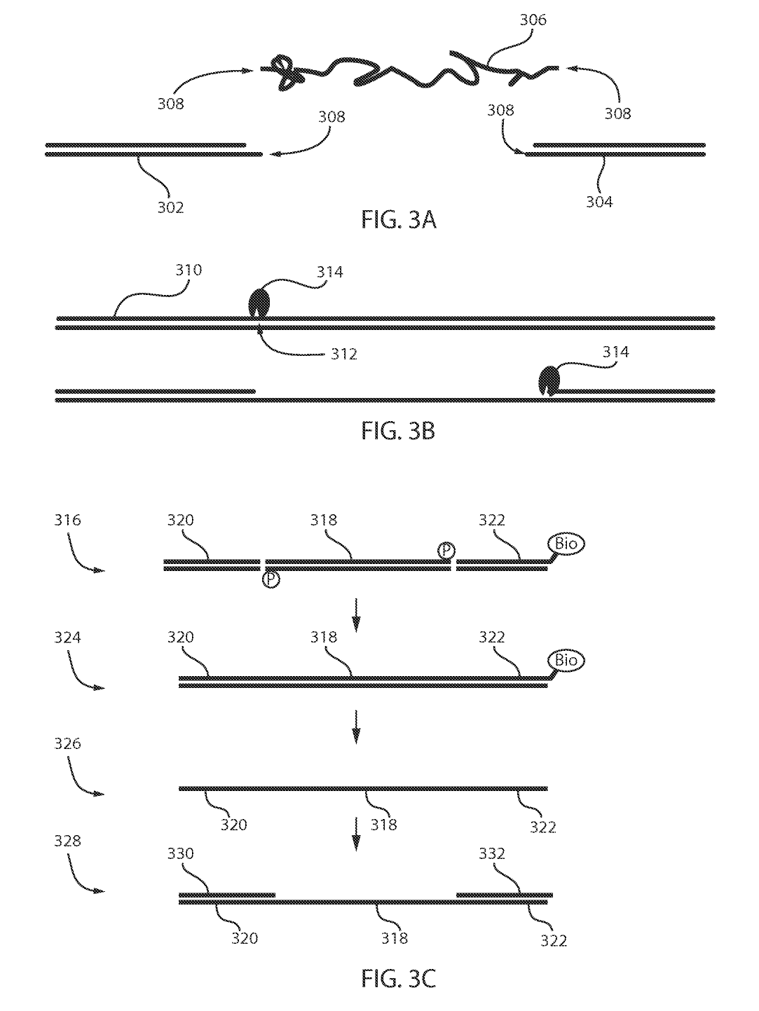

The ds-ss hybrid polynucleotide chain may be designed using a number of biochemical approaches. Referring now to FIG. 3A, a ds-ss hybrid polynucleotide chain is formed by ligation in accordance with one illustrative embodiment. The ds segments 302, 304 can be modified with sticky ends 308 on a first end and a non-phosphorylated blunt end on a second end. The ss segment 306 may be modified with stick ends 308 on both ends. The ds segments 302, 304 and ss segment 306 may be ligated to form the hybrid polynucleotide chain. Preferably, the sticky ends 308 each have different bases: adenine (A), thymine (T), guanine (G), and cytosine (C).

Referring now to FIG. 3B, a ds-ss hybrid polynucleotide chain is formed by cutting in accordance with one illustrative embodiment. A ds nucleotide chain 310 includes recognition sites 312. The recognition sites 312 can be cut by enzymes 314, such as, e.g., nicking endonuclease, to form an ss segment from the ds polynucleotide chain.

Referring now to FIG. 3C, a ds-ss hybrid polynucleotide chain is formed by random sequencing using a modified Roche 454 approach, in accordance with one illustrative embodiment. In configuration 316, ds polynucleotide segments 318 with phosphorylated ends can be ligated to ds adaptors 320, 322. The adaptors 320, 322 differ by the presence of a biotin tag on the adaptor 322. The adaptors 320, 322 may have the same or different nucleotide sequences. In configuration 324, after ligation, nicks are present at the junctions of each adaptor. The nicks are filled by the strand-displacement activity of a polymerase (e.g., DNA polymerase). This forms ds polynucleotide segments 318 with ds segments 320, 322, and a biotin attached on adaptor 322. For example, where the ds polynucleotide segments 318 are DNA, the configuration may be adaptor 320-DNA-adaptor 322-biotin. Other configurations may also be employed, e.g., adaptor 320-DNA-adaptor 320 or biotin-adaptor 322-DNA-adaptor 322-biotin. In configuration 326, ds polynucleotide segments 318 are bound to, e.g., Streptavidin beads through the biotin tagged on adaptor 322, and unbound fragments are washed away. The immobilized fragments are then denatured to split the ds portions into ss. Both bound strands remain immobilized through the biotinylated adaptor 322, while only ss fragments are washed free and used in subsequent sequencing steps. In configuration 328, the complementary strands 330, 332 for adaptors 320, 322 can be added to form a ds-ss mixed polynucleotide chain from a genomic random polynucleotide.

In another embodiment, the ds segments in configuration 328 are not formed by adding complementary adaptor strands, but instead by hybridization of the adaptors themselves (e.g., by annealing) to self-form complementary adaptors.

In still another embodiment, the biotinylated single-stranded segments in configuration 326 that bind to streptavidin-linked beads are also collected together with the beads after denaturation. Then complementary adaptor strands are added to form the double-stranded adaptors on the beads, and finally the double-stranded single-stranded mixed DNA fragments are collected by cutting the biotin-streptavidin binding.

Other approaches to forming a ds-ss hybrid polynucleotide may also be employed within the context of the present principles. For example, in one embodiment, a ds-ss hybrid polynucleotide chain may be modified to attach the sticky ends and ligated to another ss polynucleotide chain, ds polynucleotide chain, or a ds-ss hybrid polynucleotide chain. In another embodiment, a ds-ss hybrid polynucleotide chain may be cut by enzymes multiple times to form multiple ss segments.

An ss polynucleotide chain has been found to behave very differently than a ds-ss hybrid polynucleotide chain in a confined volume. The volume may include, e.g., a nanofluidic device, such as a nanochannel or a nanopore. The nanochannel or nanopore can be fabricated using, e.g., insulating or semiconducting materials, such as silicon dioxide (SiO₂), silicon nitride (Si₃N₄), alumina (Al₂O₃), titanium dioxide (TiO₂), silicon (Si), organic polymers, etc., or a combination thereof.

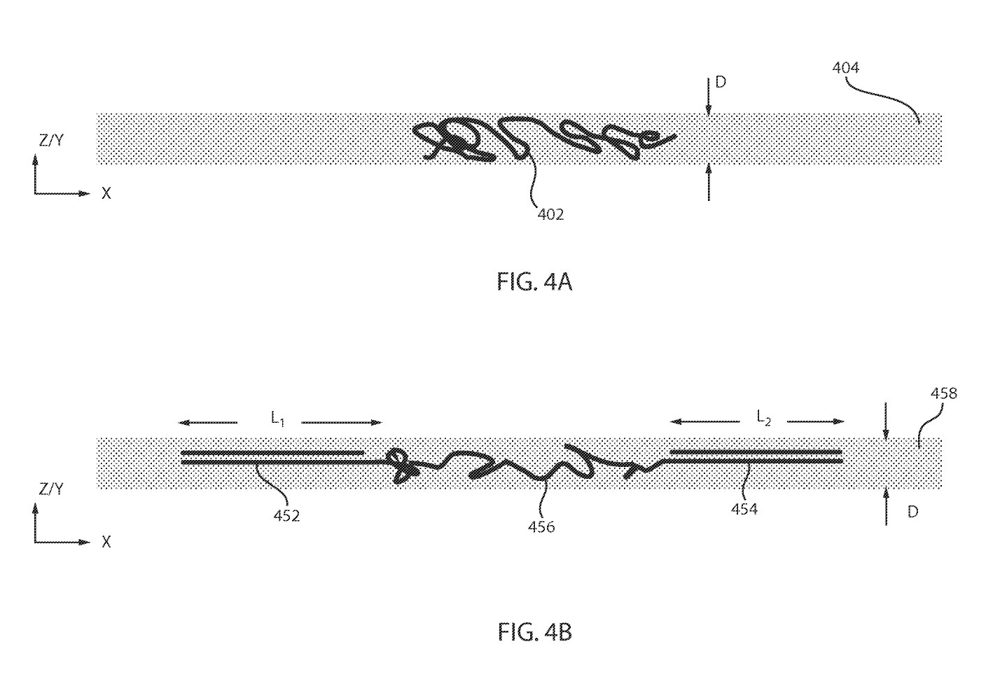

Referring now to FIG. 4A, an ss polynucleotide in a volume is illustrative depicted in accordance with one illustrative embodiment. An ss polynucleotide chain 402 is confined in volume 404 having a diameter D. Where, e.g., 5 nm<D<50 nm or D>50 nm, the ss polynucleotide chain 402 coils randomly without any control as to the position of the chain ends. The ss segments may form secondary structures by binding its different segments together using, e.g., hydrogen bonds, hydrophobic forces, etc. The binding further complicates the structures of the ss polynucleotide chain 402.

Referring now to FIG. 4B, a ds-ss hybrid polynucleotide in a volume is illustratively depicted. The ds-ss hybrid polynucleotide is confined in the volume 458 having a diameter D. Where D is smaller than about, e.g., 50 nm, the ds segments 452, 454 fully stretch no matter how long contour lengths L₁ and L₂ are. Inside such a confined volume 458, the electrostatic repulsion and the excluded volume effect drive the two ds segments 452, 454 away from each other, essentially orienting the polynucleotide chain. The ds segments 452, 454 do not have strong interaction with the ss segment 456, and thus do not cause any more complicated secondary structures.

Where D is larger than, but not much greater than about, e.g., 50 nm (e.g., 100-200 nm) and L₁ and L₂ are both smaller than about, e.g., 100 nm, the ds segments 452, 454 still fully stretches. Inside such a reduced confinement of volume 458, the electrostatic repulsion and the excluded volume effect are less strong but may be enough to keep the ds segments 452, 454 away from each other.

Where D is larger than, but not much greater than about, e.g., 50 nm (e.g., 100-200 nm) and L₁ and L₂ are much larger than 100 nm (e.g. larger than 500-1000 nm), the ds segments 452, 454 will coil. The long ds segments 452, 454 lead to larger electrostatic repulsion and are likely to keep the ds segments 452, 454 away from each other.

Where D is much larger than about, e.g., 50 nm, the ds segments 452, 454 may stretch if L₁ and L₂ are smaller than 100 nm or coil if L₁ and L₂ are larger than about, e.g., 100 nm. Since there is minimal confinement, the polynucleotide chain is very likely to coil.

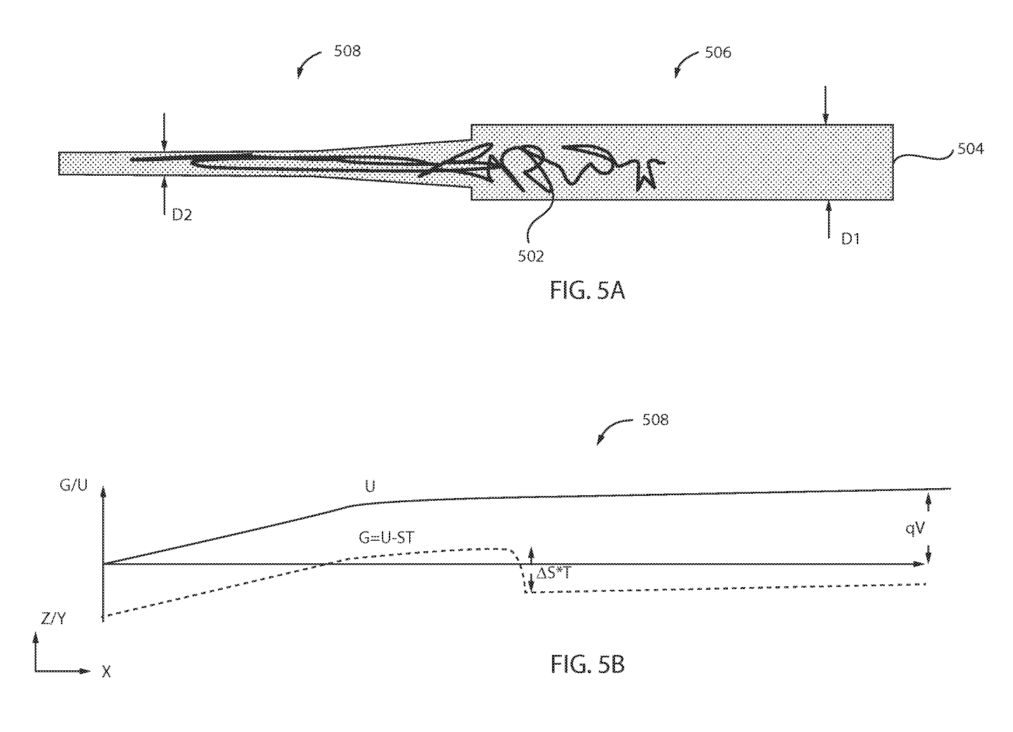

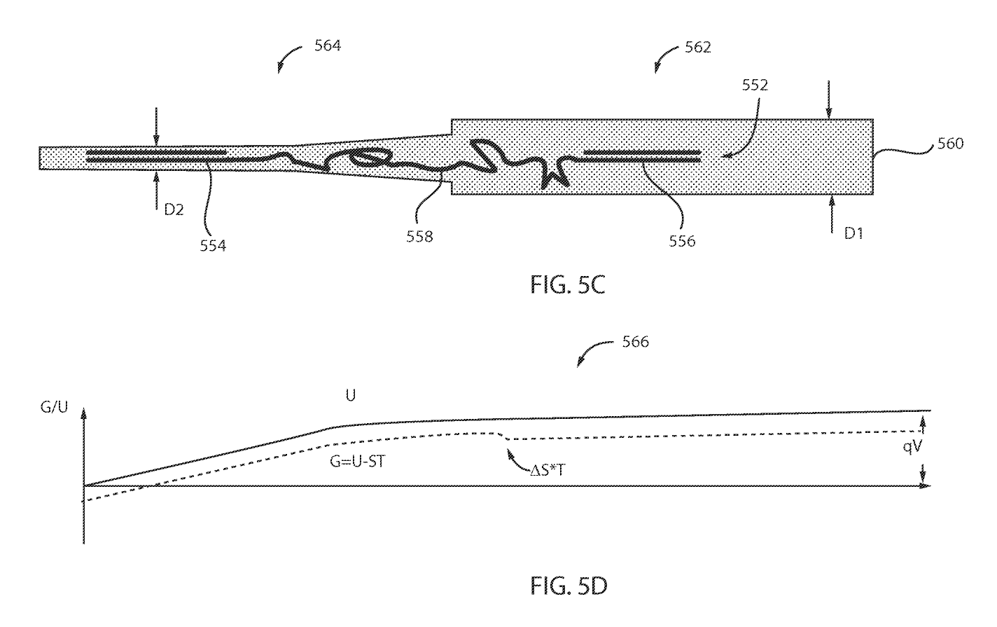

Referring now to FIG. 5A, an ss polynucleotide chain in a volume having varying regions of confinement is illustratively depicted in accordance with one embodiment. An ss polynucleotide chain behaves very differently from the ds-ss hybrid polynucleotide chain when entering from a less confined space to a more confined space, such as in a nanochannel. The ss polynucleotide chain 502 is confined in volume 504. The volume 504 has less confined regions 506 with diameter D1 and more confined or narrower regions 508 with diameter D2, where D1>D2. The less confined regions 506 may include, e.g., a micro-patterned wider channel or a non-patterned bulk solution region and the more confined regions 508 may include, e.g., nanochannels or nanopores.

In one embodiment, where D2 is smaller than about, e.g., 5 nm, the ss polynucleotide chain 502 may enter the narrower regions 508 in a stretched state. It is also possible that the ss polynucleotide chain 502 may get stuck at the interface of the transition region and does not enter.

In another embodiment, where D2 is much greater than about, e.g., 5 nm, the ss polynucleotide chain 502 is expected to translocate with a small rate. This is because the ss nucleotide chain 502 experiences a large entropic barrier to enter. Such an entropic barrier makes the ss polynucleotide chain 502 very likely to enter into the narrow regions 508 still being coiled. This can greatly enhance the friction force of the ss polynucleotide chain 502 and the volume 504 sidewall, which may cause the ss polynucleotide 502 to get stuck into the narrow region 508. This will also cause undesirable readings of multiple bases on the folded chain, and the reading can start anywhere in the chain rather than the head or the tail. The small persistence length makes the ss polynucleotide chain 502 in the narrower regions 508 easily deformed.

Referring for a moment to FIG. 5B, a graph 508 shows electrostatic energy (U) and the Gibbs free energy (G=U−ST) of an ss polynucleotide chain, where T represents the thermodynamic temperature in an absolute scale, e.g., Kelvin, and S represents the entropy of the ss polynucleotide along the channel. In the graph, qV is indicated where q is charge and V is voltage. Also, AST is shown representing the entropic barrier.

Referring back to FIG. 5A, in another embodiment, where D2 is smaller than about, e.g., 5 nm, the ss polynucleotide chain 502 may enter into the narrower regions 508 being stretched. At the same time, the entropic barrier is even higher, and thus the translocation rate is even smaller. It is also possible that the ss polynucleotide chain 502 gets stuck at the interface of the transition region and does not enter, because its coiled state (and hence larger surface area) may interact with the surface of the narrower regions 508 more strongly and hinder the entrance.

Referring now to FIG. 5C, a ds-ss hybrid polynucleotide chain in a volume having varying regions of confinement is illustratively depicted in accordance with one embodiment. The ds-ss polynucleotide chain 552 is shown in volume 560 having less confined regions 562 with diameter D1 and more confined or narrower regions 564 with diameter D2. When D2 is smaller than about, e.g., 50 nm, the ds-ss polynucleotide chain 552 is expected to stretch. Since the ds segments 554, 556 are rigid, they experience a very small entropic barrier, and thus are much easier to enter. This allows a much more controllable entry direction control.

Referring for a moment to FIG. 5D, a graph 566 shows electrostatic energy and the Gibbs free energy of the ds-ss polynucleotide chain. The entropic barrier (AST) is shown to be small.

Referring back to FIG. 5C, in another embodiment, D2 can be gradually changed from about, e.g., 50 nm to smaller than about, e.g., 5 nm, and preferably, e.g., 3-4 nm. Therefore, the ds polynucleotide head segment 554 enters the narrower regions 564 first and pulls the ss segments 558 through. As the ss segments 558 go into the narrowest region, they are forced to linearize and pass sequentially. This essentially allows the controlled sequential reading of the ss polynucleotide segments 558.

The sidewalls of the narrower regions 564 may be coated with chemicals, such as, e.g., self-assembled monolayers. The coated chemicals can be designed to interact with the ss bases in such a way that the friction to the ss segments 558 lowers the translocation speed. Such additional friction force may also stretch the ss segments 558, given the ds segment head 554 keeps pulling the chain forward.

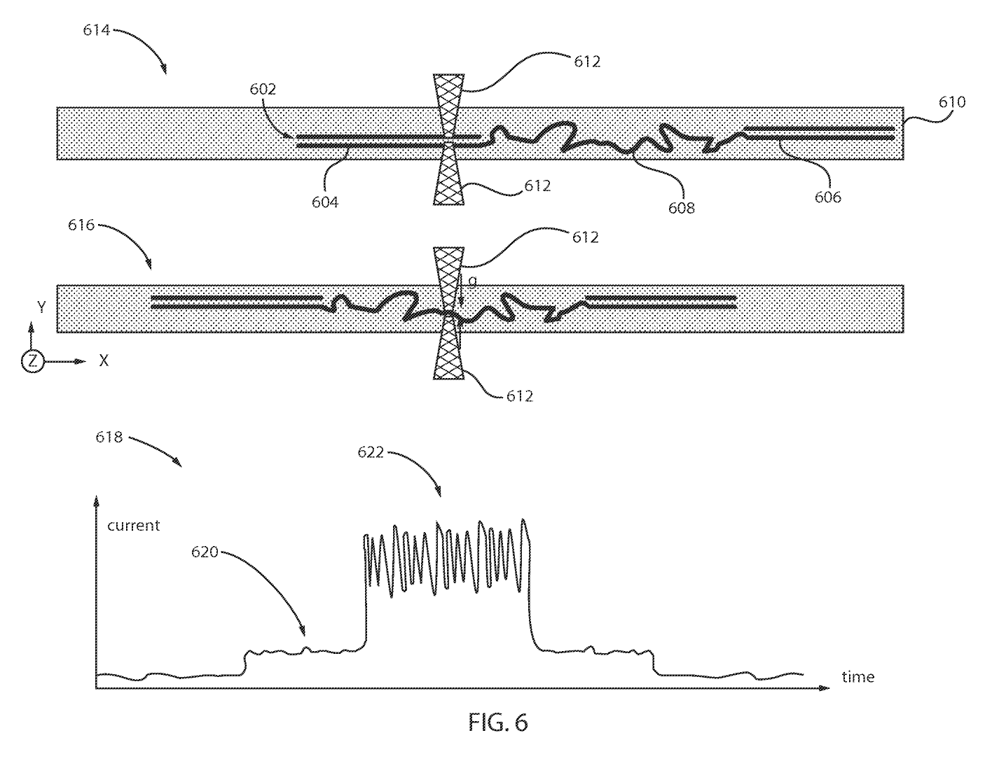

Referring now to FIG. 6, a ds-ss hybrid polynucleotide chain is electrically detected in accordance with one illustrative embodiment. The ds-ss hybrid polynucleotide chain 602 may be detected using, e.g., a pair of traverse electrodes 612. The ds-ss hybrid polynucleotide chain 602 is preferably detected in a more confined region, such as, e.g., a nanochannel or nanopore. The traverse electrodes 612 preferably include a metal, such as, e.g., gold (Au), palladium (Pd), silver (Ag), platinum (Pt), aluminum (Al), etc., a doped or conductive semiconductor, such as, e.g., silicon (Si), gallium nitrogen (GaN), etc., or a combination thereof. The traverse electrodes 612 may be coated with chemicals to chemically bind to the ss polynucleotide bases for tunneling reading.

The translocation of the ds-ss hybrid polynucleotide chain 602 produces electrical signals that can distinguish the ss segments 608 and the ds segments 604, 606. The ds segments 604, 606 lack active nucleotide bases, which can directly bind to the reading chemicals on the electrodes, and thus contributes to small signals that do not allow base discrimination. The ss segments 608 can actively interact with the reading molecules and contribute to the signals to allow distinguishing of the bases. The graph 618 shows the current signal from the electrodes 612 over time. The electrode 612 evaluating the ds segment 604 at position 614 yields signal 620. The electrode 612 evaluating the ss segment 608 at position 616 yields signal 622.

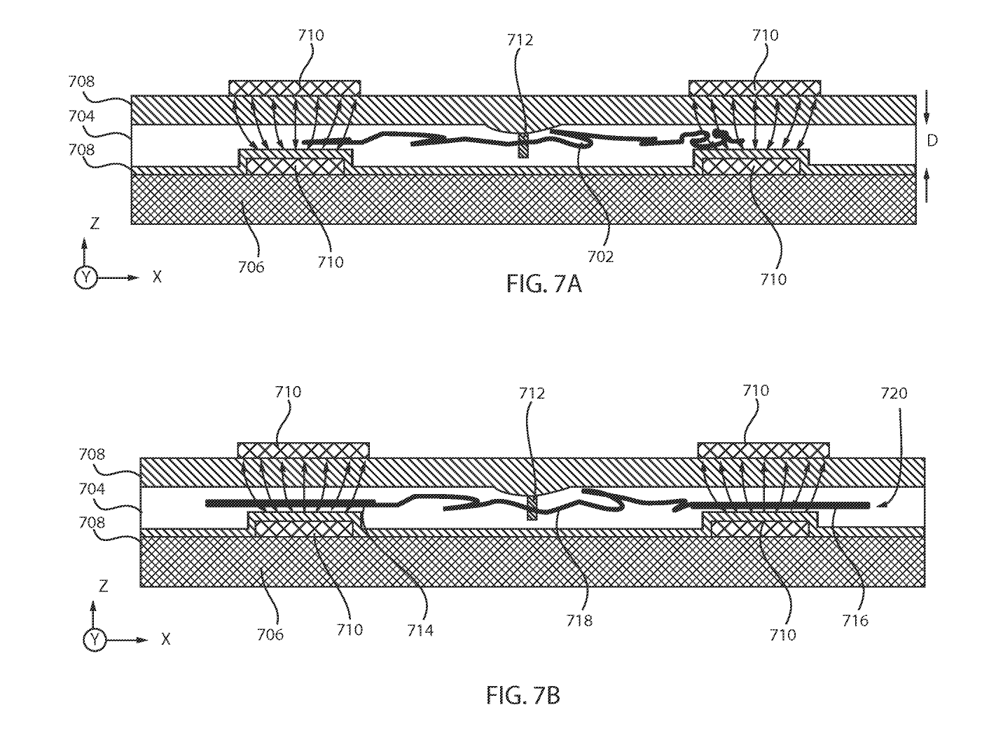

The ds-ss hybrid polynucleotide chain has a stronger interaction with applied electrostatic potentials and, thus, is easier to control than an ss polynucleotide chain. Referring now to FIG. 7A, an ss polynucleotide chain is shown in a volume having trapping electrodes integrated therein. The ss polynucleotide chain 702 is in a volume 704 having trapping electrodes 710. The volume 702 is built on a substrate 706 with insulating coating 708 for controlling the polynucleotide motion. Transverse sensing electrodes 712 may be optionally added into the volume 702.

Referring now to FIG. 7B, a ds-ss hybrid polynucleotide chain 720 having ds segments 714, 716 and ss segments 718 is in the volume 704. Where diameter D of volume 704 is smaller than about, e.g., 50 nm, the ds-ss hybrid polynucleotide chain 720 is stretched, whereas the ss polynucleotide chain 702 remains coiled (FIG. 7A). The ds segments 714, 716 have twice the charge and hence experience twice as strong electrostatic force, which is used to push the polynucleotide chain 720 against the wall of the volume 704 to control the speed of the polynucleotide based on the friction force. The stretched state of the ds-ss hybrid polynucleotide 720 yields more uniform charge distribution and hence experiences more uniformly applied electrostatic force, which is favorable to precisely control the motion of the polynucleotide chain 720.

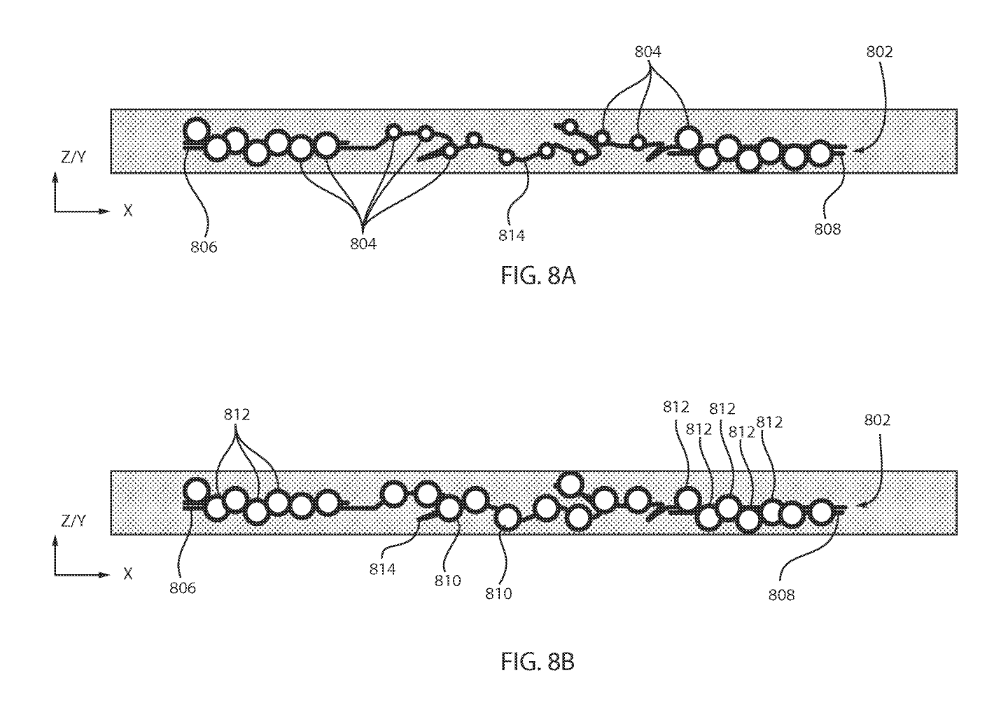

Referring now to FIGS. 8A and 8B, the ds-ss hybrid polynucleotide chain can be labeled with fluorescent dyes for optical imaging, in accordance with one illustrative embodiment. In FIG. 8A, the ds-ss hybrid polynucleotide chain 802 can be labeled with fluorescent dyes 804. The fluorescent dyes 804 may include, e.g., bisbenzimide or indole-derived stains (e.g., Hoechst 33342, Hoechst 33258), phenanthridinium stains (e.g., ethidium bromide, propidium iodide), cyanine dyes (e.g., Pico Green, YOYO-1 iodide, SYBR Green I, SYBR Gold), etc. The ds segments 806, 808 can bind better to those fluorescent dyes and yield brighter fluorescent signals. Therefore, the ds segments 806, 808 allow for more reliable detection of the ss segment 814 motion using, e.g., an optical microscope.

In FIG. 8B, the ds-ss hybrid polynucleotide chain can be labeled with multiple fluorescent dyes which can emit light of different wavelengths when binding to ss segments 814 (e.g., dye 810 emitting a first color) or ds segments 806, 808 (e.g., dye 812 emitting a second color). The dyes may include, e.g., acridine orange. Therefore, the signals of the ss segments 814 and the ds segments 806, 808 from the same polynucleotide chain can be analyzed using the same optical channel or separately, providing more information on the different segments in an independent and flexible way.

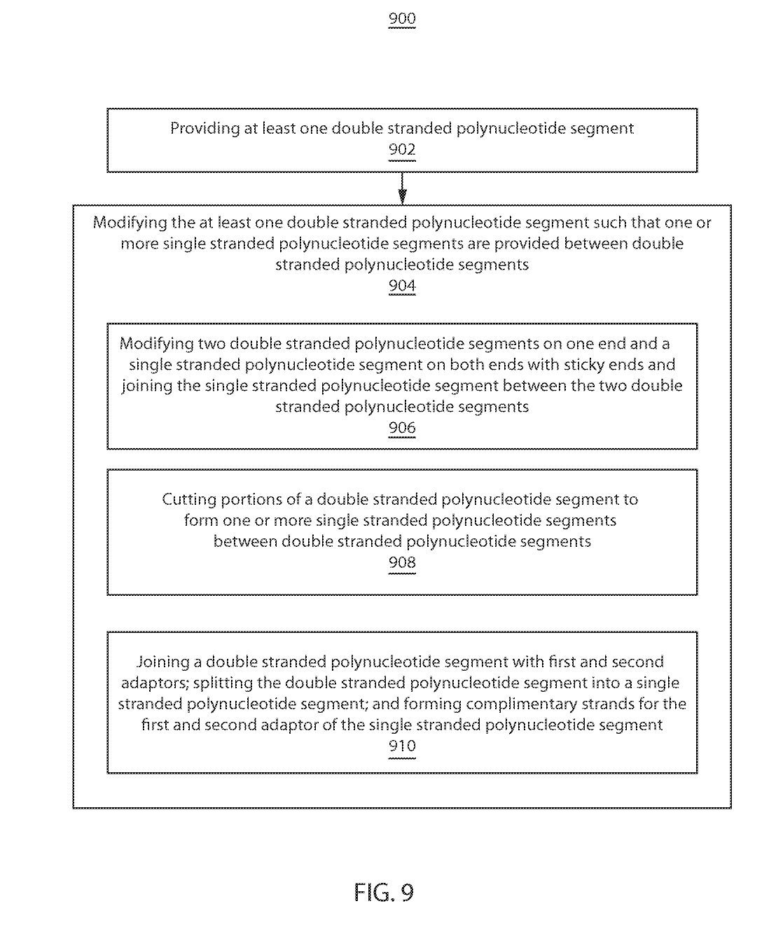

Referring now to FIG. 9, a block/flow diagram showing a method for polynucleotide formation is illustratively depicted in accordance with one embodiment. In block 902, at least one double stranded polynucleotide segment is provided. The ds polynucleotide segment may include, e.g., DNA, DNA-RNA complementary chain, etc.

In block 904, at least one double stranded polynucleotide segment is modified such that one or more single stranded polynucleotide segments are provided between double stranded polynucleotide segments. In block 906, modifying may include modifying two double stranded polynucleotide segments on one end and a single stranded polynucleotide segment on both ends with sticky ends and joining the single stranded polynucleotide segment between the two double stranded polynucleotide segments. In block 908, modifying may also include cutting portions of a double stranded polynucleotide segment to form one or more single stranded polynucleotide segments between double stranded polynucleotide segments. In block 910, modifying may further include joining at least one ds polynucleotide segment with first and second adaptors, splitting the at least one ds polynucleotide segment into at least one ss polynucleotide segment, and forming complementary strands for the first and second adaptor of the at least one ss polynucleotide segment. Forming complementary strands may include adding complementary strands, annealing to self-form complementary strands, etc.

Having described preferred embodiments of a system and method a polynucleotide configuration for reliable electrical and optical sensing (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A method for forming a polynucleotide, comprising:
forming sticky ends on at least one end of a plurality of double stranded (ds) polynucleotide segments and one or more single stranded (ss) polynucleotide segments; and
joining the sticky ends of the one or more ss polynucleotide segments between the plurality of ds polynucleotide segments to provide a mixed ds-ss polynucleotide.

2. The method as recited in claim 1, wherein joining includes ligating the one or more ss polynucleotide segments between the plurality of ds polynucleotide segments.

3. The method as recited in claim 1, wherein the sticky ends have different bases.

4. The method as recited in claim 1, further comprising confining the mixed ds-ss polynucleotide in a volume.

5. The method as recited in claim 4, wherein the volume includes at least one of a nanochannel and a nanopore.

6. The method as recited in claim 4, further comprising detecting the mixed ds-ss polynucleotide in the volume such that ds segments and ss segments are distinguished.

7. The method as recited in claim 4, further comprising employing electrodes in the volume to control a motion of the mixed ds-ss polynucleotide by electrostatically pushing the mixed ds-ss polynucleotide against a wall of the volume

8. The method as recited in claim 1, further comprising labeling the mixed ds-ss polynucleotide with one or more fluorescent dyes.

9. The method as recited in claim 8, wherein ds segments of the mixed ds-ss polynucleotide have brighter fluorescence than ss segments.

10. The method as recited in claim 8, wherein ds segments of the mixed ds-ss polynucleotide emit light having a different wavelength than ss segments.

11. The method as recited in claim 1, wherein the mixed ds-ss polynucleotide includes a plurality of ds and ss segments formed between the plurality of ds polynucleotide segments.

12. The method as recited in claim 1, wherein the mixed ds-ss polynucleotide includes at least one of DNA and RNA.
